(19) United States
(12) Patent Application Publication
(10) Pub. No.: US 2009/0026565 A1
(43) Pub. Date: Jan. 29, 2009

(54) Optical Module

(76) Inventors: Arihide Noda, Tokyo (JP); Mikio Oda, Tokyo (JP); Takashi Ohtsuka, Tokyo (JP); Hisaya Takahashi, Tokyo (JP); Hikaru Kouta, Tokyo (JP); Jun Sakai, Tokyo (JP)

Correspondence Address: MCGINN INTELLECTUAL PROPERTY LAW GROUP, PLLC, 8321 OLD COURTHOUSE ROAD, SUITE 200, VIENNA, VA, 22182-3817, US

(21) Appl. No.: 12/223,541

(22) PCT Filed: Feb. 2, 2007
(86) PCT No.: PCT/JP2007/051766

§ 371 Date: Aug. 1, 2008

(30) Foreign Application Priority Data
Feb. 2, 2006 (JP) 2006-025894

(51) Int. Cl. H01L 31/0232; C12Q 1/68; G01J 1/00

(52) U.S. Cl. 257/432; 250/200; 250/336.1; 257/E31.127

(57) ABSTRACT

The present invention includes: photoelectric conversion element 103 that converts electrical signals into optical signals and optical signals into electrical signals; and optical communication LSI 102 electrically connected to photoelectric conversion element 103. Also, the present invention includes electrical wiring substrate 101 including a plurality of electrodes 201 and 202 on which photoelectric conversion element 103 and optical communication LSI 102 are mounted by flip-chip attachment and a plurality of wiring layers 101a, 101b and 101c electrically connecting respective electrodes 201 and 202, wiring layers 101a, 101b and 101c being provided at an upper surface, a lower surface and an inner portion of electrical wiring substrate 101, respectively. Also, electrodes 201 and 202 to which photoelectric conversion element 103 is bonded are provided at a side surface of electrical wiring substrate 101.

### TECHNICAL FIELD

The present invention relates to an optical module for converting electrical signals into optical signals and optical signals into electrical signals.

### BACKGROUND ART

In an optical interconnection, an electrical signal output from a large-scale integration circuit (LSI) is converted to an optical signal and transmitted, and after it is transmitted as an optical signal, this optical signal is converted to an electrical signal and the electrical signal is conveyed to another LSI. In recent years, the speed of signals handled by LSIs has been further increased, and also, 1000 input/output signal channels or more are provided in many cases. Consequently, there is a demand for further increasing the speed and mounting density for optical modules used in optical interconnection.

FIG. 1 is a-schematic diagram illustrating a typical conventional optical module. As shown in FIG. 1, a conventional optical module includes photoelectric conversion element 503 that converts electrical signals into optical signals and optical signals into electrical signals, optical communication LSI 502 electrically connected to photoelectric conversion element 503, another electronic component 504, and electrical wiring substrate 501 having these photoelectric conversion element 503, optical communication LSI 502 and the other electronic component 504 mounted thereon.

On the upper surface of electrical wiring substrate 501, a wiring layer forming a wiring pattern is provided, and optical communication LSI 502, photoelectric conversion element 503 and the other electronic component 504 are mounted on this wiring layer. The respective components are electrically connected by means of bonding wires 510 to electrodes (not shown) provided at the wiring layer on the upper surface of electrical wiring substrate 501, and bonding wires 510 are also used as signal interfaces between the components. Optical signals are input/output via optical wiring 505 provided above photoelectric conversion element 503.

Instead of this wire-bonding mounting, Japanese Patent Laid-Open No. 2002-217234 discloses a configuration in which a photoelectric conversion element with a flip-chip structure is bonded to an electrical wiring substrate by a bump that intervenes between the substrate and the element.

FIG. 2 is a schematic diagram illustrating a conventional optical module employing the wire-bonding mounting process in FIG. 1 and that has been changed to a flip-chip attachment (FCA) process. As a result of employment of a flip-chip attachment process, this optical module enables reduction of stray capacitance and inductance caused by the wirings, and thus it is suitable for cases where higher speed signals are handled.

As shown in FIG. 2, LSI 502 and photoelectric conversion element 503 mounted on electrical wiring substrate 501 are electrically connected to electrodes of electrical wiring substrate 501 by bumps 607 that intervenes between the electrodes and the element, and signals between the components are electrically connected via an upper surface wiring layer, a lower surface wiring layer and an inner wiring layer provided on the upper surface, the lower surface and an inner portion of electrical wiring substrate 501, respectively.

Other examples of a configuration in which optical signals are input/output by means of the configuration, which is mentioned above, include a configuration in which a photoelectric conversion element that receives/emits optical signals via through holes formed in an electrical wiring substrate is provided on one surface of the electrical wiring substrate and is connected to optical wirings arranged on the other surface, and a configuration in which a photoelectric conversion element that receives/emits light is arranged on a surface opposite the surface on which electrodes bonded to an LSI are provided and is connected to an optical wiring.

### DISCLOSURE OF THE INVENTION

As described above, in a conventional optical module for optical interconnection, an optical communication LSI, a photoelectric conversion element and another electronic component are respectively mounted on an upper surface wiring layer of an electrical wiring substrate, and a wiring layer is provided only at the upper and lower surface and an inner portion of the electrical wiring substrate.

Since the conventional optical module shown in FIG. 1 employs wire-bonding mounting process, it is necessary to arrange the electrodes on the upper surface of the electrical wiring substrate and outside the outer periphery of the electronic components such as the optical communication LSI. Consequently, the electrical wiring substrate needs to have a large area. Also, as a result of the surface for mounting the components being limited to the upper surface of the electrical wiring substrate, the electrical wiring substrate necessarily has an area larger than the area occupied by the components, which is unsuitable for high-density mounting. Also, inductance components, etc., in the wires cause impedance mismatching or electrical signal attenuation, therefore, making high-speed signal transmission difficult.

In the conventional optical module shown in FIG. 2, since no wires are used for the electrical connection between the components and the electrodes, the electrodes on the electrical wiring substrate can be arranged immediately below the components, making it possible to provide an area of the electrical wiring substrate that is smaller compared to that of the conventional optical module shown in FIG. 1.

However, in this conventional optical module, as in the conventional optical module shown in FIG. 1, the surface for mounting the components is limited to the upper surface of the electrical wiring substrate, and accordingly, it is unsuitable for further increasing the mounting density. Also, this conventional optical module does not use wires as electric wirings for electrical connection with the electrodes, and accordingly, transmission band deterioration due to inductance components, etc., can be prevented. However, wiring layers for electrically connecting the components are necessarily provided at the upper surface, the lower surface and the inner portion of the electrical wiring substrate, resulting in the disadvantage of band limitation being generated due to stray capacitances between the respective wiring layers.

Furthermore, the electrodes for mounting an LSI have a width larger than the width of the wirings at the wiring layers, and parasitic capacitances are generated between these electrodes and other conductors. In particular, when the inner wiring layer of the electrical wiring substrate is provided with a ground layer, the stray capacitances between these layers increase further. Also, where the wirings between the LSI and the photoelectric conversion element are relatively long and have large stray capacitances, further significant band deterioration occurs. In order to increase the speed, it is necessary to minimize the stray capacitances provided to the photoelectric conversion element.

As described above, the mounting structures for the conventional optical modules have many problems in providing high-speed optical interconnection.

Therefore, an object of the present invention is to provide an optical module that enables high-density mounting, optical module downsizing, and increasing the speed of signal transmission.

In order to achieve the object which is mentioned above, an optical module according to the present invention includes: a photoelectric conversion element that converts electrical signals into optical signals and optical signals into electrical signals; and an optical communication integrated circuit electrically connected to the photoelectric conversion element. Also, this optical module includes an electrical wiring substrate including a plurality of electrodes on which the photoelectric conversion element and the optical communication integrated circuit are mounted by flip-chip attachment, and a plurality of wirings electrically connecting the respective electrodes, and in which the wirings are provided at an upper surface, a lower surface and an inner portion of the electrical wiring substrate. The electrodes to which the photoelectric conversion element is bonded are provided at a side surface of the electrical wiring substrate.

According to the optical module according to the present invention configured as described above, as a result of the photoelectric conversion element being mounted on the side surface of the electrical wiring substrate by flip-chip attachment, the area of the electrical wiring substrate can be minimized and the electronic components can be mounted at a high density. Accordingly, optical module downsizing can be achieved.

Also, according to this optical module, as a result of the photoelectric conversion element being mounted on the side surface of the electrical wiring substrate, the lengths of the wirings between the optical modules that perform signal transmission can be reduced. Thus, attenuation due to loss in the wirings or stray capacitances generated between the respective wirings in the electrical wiring substrate can be reduced, and band deterioration due to impedance mismatching or electrical signal attenuation, etc., can also be minimized.

Also, it is preferable that the plane formed by the electrodes at the side surface and the wirings at the side surface included in the optical module according to the present invention be perpendicular to the wirings and to the inner layer of the electrical wiring substrate. As a result, parasitic capacitances generated between the electrodes at the side surface and the wirings at the side surface, and the wirings and the inner layer of the electrical wiring substrate can be minimized, enabling prevention of band deterioration. Accordingly, high-speed signal transmission can easily be performed, enabling an increase in optical interconnection speed.

Also, an engagement pin for aligning an optical wiring to be connected to the photoelectric conversion element, and the photoelectric conversion element with each other, may be provided at the side surface of the electrical wiring substrate included in the optical module according to the present invention. As a result, the optical wiring and the photoelectric conversion element are aligned with each other with high accuracy, enabling suppression of optical coupling loss.

Also, a reference portion for positioning a light-emitting portion or a light-receiving portion of the photoelectric conversion element on the electrical wiring substrate may be provided at a corner between the side surface and the upper surface of the electrical wiring substrate included in the optical module according to the present invention. As a result, the photoelectric conversion element is positioned on the electrical wiring substrate with high accuracy, resulting in highly-accurate optical coupling of the optical wiring and the photoelectric conversion element.

As described above, according to the present invention, the area of the electrical wiring substrate can be minimized and the photoelectric conversion element and the integrated circuit can be mounted on the electrical wiring substrate at a high density, enabling optical module downsizing. Thus, according to the present invention, signal attenuation can be minimized by parasitic capacitance reduction or loss due to reduction of the wiring lengths of the electrical wiring substrate, and also, as a result of side surface mounting, parasitic capacitances generated in these electrodes or wirings can be reduced. As a result, band deterioration can be suppressed and the signal transmission speed can easily be increased, enabling an increase in optical interconnection speed.

### BRIEF DESCRIPTION OF THE DRAWINGS

FIG. 1 is a diagram illustrating a structure employing wire-bonding mounting process for wirings as an example of a conventional optical module;

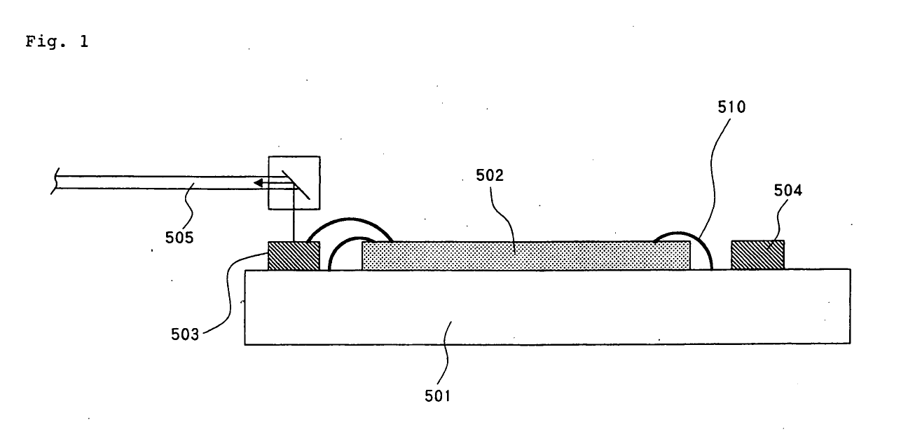

FIG. 2 is a diagram illustrating a structure employing flip-chip attachment process that uses bumps for wirings as another example of a conventional optical module;

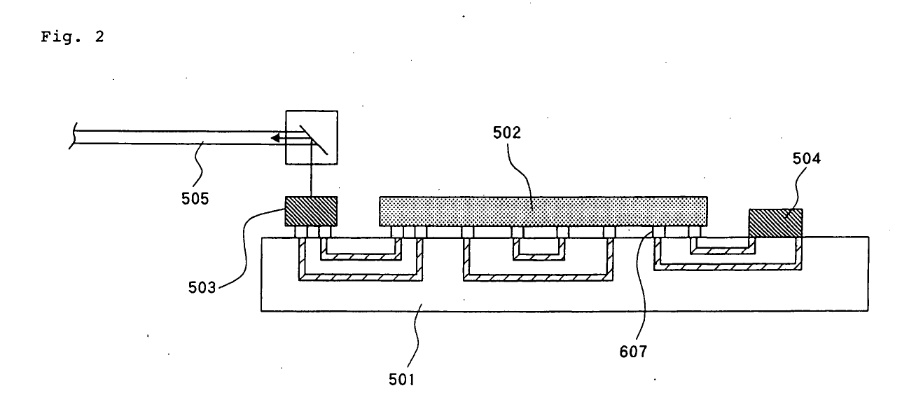

FIG. 3 is a schematic diagram illustrating an optical module according to a first exemplary embodiment;

FIG. 4 is a perspective view showing wirings and electrodes of an electrical wiring substrate included in the optical module according to the first exemplary embodiment;

FIG. 5 is a schematic diagram illustrating an optical module according to a second exemplary embodiment; and

FIG. 6 is a schematic diagram illustrating an optical module according to a third exemplary embodiment.

### BEST MODE FOR CARRYING OUT THE INVENTION

Hereinafter, specific exemplary embodiments of the present invention will be described with references to the drawings.

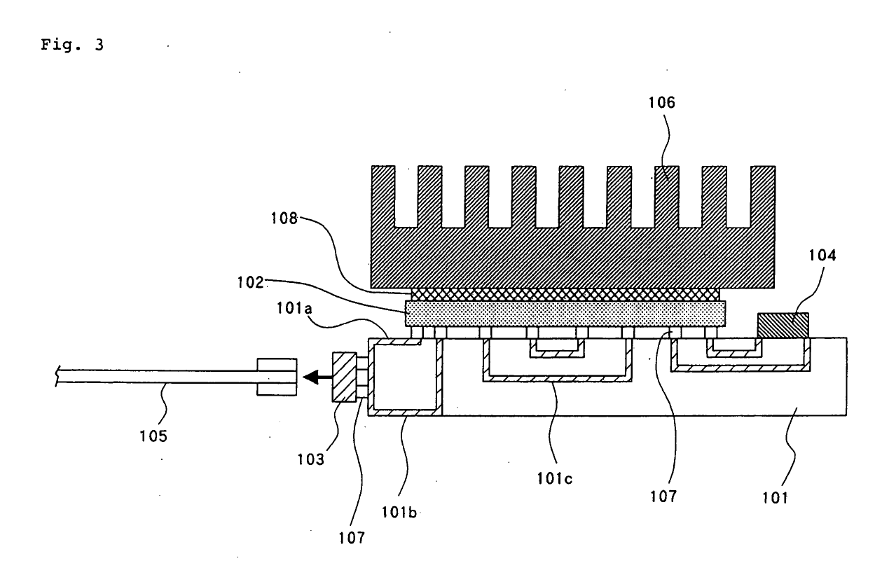

As shown in FIG. 3, an optical module according to an exemplary embodiment includes photoelectric conversion element 103 that converts electrical signals into optical signals and optical signals into electrical signals, optical communication large-scale integration circuit (LSI) 102 electrically connected to photoelectric conversion element 103, another electronic component 104, and electrical wiring substrate 101 on which photoelectric conversion element 103, LSI 102 and the other electronic component 104 are mounted by means of flip-chip attachment.

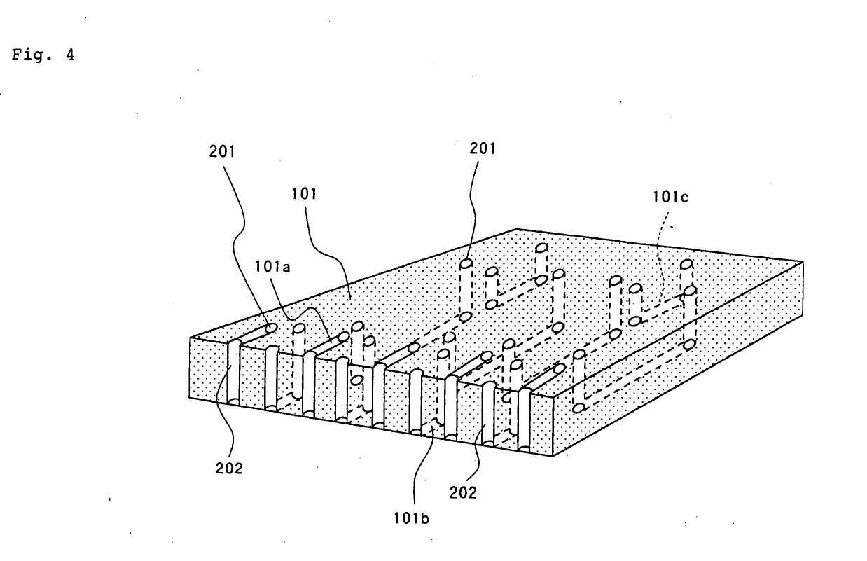

As shown in FIG. 4, electrical wiring substrate 101 is a multilayer wiring substrate in which upper surface wiring layer 101a and lower surface wiring layer 101b, which each having a desired wiring pattern, are formed at both surfaces, that is, the upper and lower surface of a base material formed of ceramics or other materials, and plurality of inner wiring layers 101c each having a desired pattern is provided at an inner portion, in parallel to upper surface wiring layer 101a and lower surface wiring layer 101b. Plurality of electrodes 201 is provided on the wiring at upper surface wiring layer 101a of electrical wiring substrate 101, and LSI 102 and the other component 104 are mounted on these electrodes 201 using bumps 107 by means of flip-chip attachment.

Also, what is called through holes, which penetrate electrical wiring substrate 101 in the thickness direction, are provided in electrical wiring substrate 101, and the half-cylindrical cross sections of the through holes are formed at a side surface of electrical wiring substrate 101 by cutting electrical wiring substrate 101 along the axis lines of the through holes in the thickness direction. Using conductive films, being provided in the through hole cross sections, that is, the through hole inner surfaces, and their cross sections, as electrodes 202, photoelectric conversion element 103 is mounted on these electrodes 202 using bumps 107 at the side surface of electrical wiring substrate 101.

Also, other through holes that electrically connect the wirings in upper surface wiring layer 101a, inner wiring layer 101c and lower surface wiring layer 101b are provided in the electrical wiring substrate 101.

Linearly-arranged optical wiring 105 is optically connected to photoelectric conversion element 103 mounted on the side surface of electrical wiring substrate 101.

Also, metal radiating member 106 including a plurality of radiating fins is bonded using radiating material 108, for example, a silicone oil compound, to the upper surface of LSI 102 mounted on electrical wiring substrate 101.

As shown in FIG. 4, wirings electrically connected to side-surface electrodes 202 are provided at upper surface wiring layer 101a and inner wiring layer 101c of electrical wiring substrate 101, respectively. Accordingly, photoelectric conversion element 103, whose terminal was bonded to side-surface electrodes 202 using bumps 107, is electrically connected by the wirings to LSI 102 and the other electronic component 104, which were bonded to electrodes 201 at upper surface wiring layer 101a using bumps 107.

The structure of electrodes 202 provided at the side surface in such a manner as described above is not limited to a configuration using through hole cross sections, and a structure in which generally-used metal foil (copper foil) is attached to the side surface or a structure in which a conductive film is deposited on the side surface by plating, etc., may be used as electrodes.

A configuration in which electrodes are formed at a side surface of an electrical wiring substrate can be provided in such a manner as described above by, for example, forming in advance electrodes and multiple wiring layers at a relatively-thin electrical wiring substrate, e.g., a flexible wiring substrate, and attaching this relatively-thin electrical wiring substrate to a relatively-thick electrical wiring substrate, which is formed of, e.g., glass or organic material, so that it twists around from the upper surface to a side surface of the relatively-thick electrical wiring substrate, which is not shown.

In general, a ground layer is often provided in an inner wiring layer of an electrical wiring substrate, resulting in the disadvantage of stray capacitances, being generated between the wirings and this ground layer, causing band deterioration for signals that pass in the wirings. However, the optical module according to this exemplary embodiment, as shown in FIG. 3, the plane formed by the electrodes provided at the side surface of the electrical wiring substrate and the plane formed by the inner wiring layer (ground layer plane) of the electrical wiring substrate are in a positional relationship in which they are perpendicular to each other, and accordingly, the stray capacitance generated therebetween can be minimized, enabling band deterioration to be minimized.

In particular, where photoelectric conversion element 103 is a light-receiving element, the stray capacitance between it and LSI 102 has a great influence on band limitation. For example, where the band is no less than 10 Gbps, the properties cannot be ensured logically unless the stray capacitance provided to the wirings and to the electrodes for photoelectric conversion element (light-receiving element) 103 is made to be several tens of fF or less. Thus, it is important to reduce the wiring length to minimize stray capacitance generated between the electrodes and wirings.

As described above, according to the optical module according to this exemplary embodiment, electrodes 202 are provided at the side surface of electrical wiring substrate 101, and photoelectric conversion element 103 is mounted on the side surface by means of flip-chip attachment, whereby the area of electrical wiring substrate 101 can be minimized and photoelectric conversion element 103 and LSI 102 can be mounted on electrical wiring substrate 101 at a high density, enabling optical module downsizing.

Also, according to this optical module, LSI 102 can be mounted adjacent to the position for mounting photoelectric conversion element 103, enabling reduction of the length of the wiring between LSI 102 and photoelectric conversion element 103. Thus, in the optical module according to this exemplary embodiment, band deterioration due to loss in the wiring can be suppressed and the signal transmission speed can be easily increased, enabling an increase in optical interconnection speed.

Also, in the optical module according to the present exemplary embodiment, the density can be further increased by arranging a part of the other electronic component 104 in the inner portion of the electrical wiring substrate 101 or mounting it on a side surface or the upper or lower surface where necessary.

### SECOND EXEMPLARY EMBODIMENT

Next, a second exemplary embodiment will be described with reference to the drawings. In the second exemplary embodiment, for convenience, the members that are the same as those in the first exemplary embodiment are provided with the same reference numerals and the description thereof will be omitted.

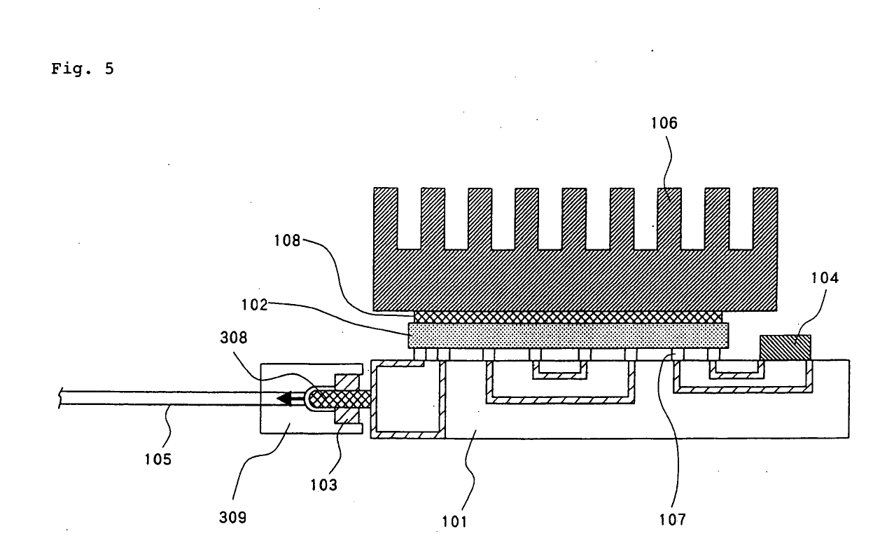

As shown in FIG. 5, an optical module according to the second exemplary embodiment is provided with engagement pin 308 for positioning the connection between optical wiring 105 and photoelectric conversion element 103 on a side surface of electrical wiring substrate 101, in addition to the configuration of the first exemplary embodiment. Also, on the optical wiring 105 side, engagement connector 309 including an engagement hole that engages with engagement pin 308 on the optical module side is provided.

In the optical module according to the present exemplary embodiment, engagement pin 308 is provided on the side surface of electrical wiring substrate 101, enhancing the accuracy of the position of the connection between optical wiring 105 and photoelectric conversion element 103, thereby enabling further suppression of optical signal attenuation due to optical coupling misalignment. Also, the optical module can have a structure in which optical wiring 105 and photoelectric conversion element 103 can be connected to/disconnected from each other by means of engagement connector 309, as a result of engagement pin 308 being provided on the side surface of electrical wiring substrate 101.

### THIRD EXEMPLARY EMBODIMENT

Lastly, a third exemplary embodiment will be described with reference to the drawings. In the third exemplary embodiment, for convenience, the members that are the same as those in the first exemplary embodiment are provided with the same reference numerals and the description thereof will be omitted.

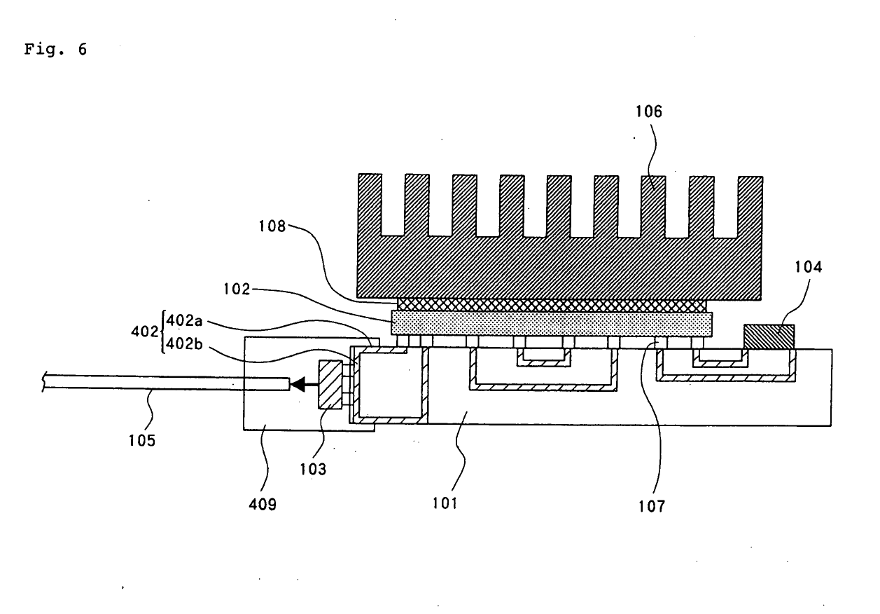

As shown in FIG. 6, in an optical module according to the third exemplary embodiment, instead of engagement pin 306 in the second exemplary embodiment, reference portion 402 for positioning a light-emitting portion or a light-receiving portion of photoelectric conversion element 103 on electrical wiring substrate 101 is formed at a corner portion between a side surface and the upper surface of electrical wiring substrate 101. This reference portion 402 includes reference upper surface 402a and reference side surface 402b, and using reference surfaces 402a and 402b of reference portion 402 of electrical wiring substrate 101 as positioning references, the position for mounting photoelectric conversion element 103 on electrical wiring substrate 101 is determined with high accuracy. Also, on the optical wiring 105 side, engagement connector 409 including an engagement hole that engages with reference portion 402 on the optical module side is provided. Reference portion 402 may be formed at a corner portion between the side surface and the lower surface of electrical wiring substrate 101.

In the optical module according to the present exemplary embodiment, photoelectric conversion element 103 is mounted after being positioned using reference portion 402 of electrical wiring substrate 101 as a positioning reference, whereby the relative position between electrical wiring substrate 101 and the light-emitting and light-receiving point of photoelectric conversion element 103 is kept constant at all times. When the positional relationship of the engagement between electrical wiring substrate 101 and optical wiring 105, and engagement connector 409 is constant all the times, correspondingly, optical signal attenuation caused by the engagement between photoelectric conversion element 103 and optical wiring 105 can further be suppressed.

In the optical module according to each of the exemplary embodiments which are mentioned above, optical wiring 105 is arranged linearly, but optical wiring 105 may be drawn in another direction by bending the optical path for optical signals using refraction means (not shown), e.g., a prism. Also, radiating member 106 may be omitted if the value of heat from LSI 102, etc., is moderate. Furthermore, it should be understood that: the other electronic component 104 mounted on electrical wiring substrate 101 is not limited to being mounted on the upper surface wiring layer of the electrical wiring substrate; and it may be mounted on the lower surface wiring layer of the electrical wiring substrate, or may also be mounted onto the electrical wiring substrate.

Also, an optical module according to the present invention is suitable for use in various types of optical communication system that transmits/receives information via, for example, an optical fiber.

1. An optical module comprising:
a photoelectric conversion element that converts electrical signals into optical signals and optical signals into electrical signals;
an optical communication integrated circuit electrically connected to the photoelectric conversion element; and
an electrical wiring substrate including a plurality of electrodes on which the photoelectric conversion element and the optical communication integrated circuit are mounted by flip-chip attachment, and a plurality of wirings electrically connecting the respective electrodes, and in which the wirings are provided at an upper surface, a lower surface and an inner portion of the electrical wiring substrate, respectively, and characterized in that
the electrodes to which the photoelectric conversion element is bonded, are provided at a side surface of the electrical wiring substrate.

2. The optical module according to claim 1, wherein planes formed by the wirings of the electrical wiring substrate, are perpendicular to a plane formed by the electrodes at the side surface.

3. The optical module according to claim 1, wherein the electrodes at the side surface are formed by a portion at which through holes, which are formed in the electrical wiring substrate, are cut in a thickness direction of the electrical wiring substrate.

4. The optical module according to claim 1, wherein the optical communication integrated circuit is bonded to the electrodes which are provided on the upper surface of the electrical wiring substrate.

5. The optical module according to claim 1, wherein another electrical wiring substrate having the electrodes and the wirings formed therein, is provided over the upper surface and the side surface of the electrical wiring substrate, thereby forming the electrodes at the side surface.

6. The optical module according to claim 1, wherein an engagement pin for aligning an optical wiring, which is connected to the photoelectric conversion element, with the photoelectric conversion element, is provided at the side surface of the electrical wiring substrate.

7. The optical module according to claim 1, wherein a reference portion for positioning a light-emitting portion or a light-receiving portion of the photoelectric conversion element on the electrical wiring substrate, is provided at a corner portion between the side surface and the upper surface of the electrical wiring substrate.

8. The optical module according to claim 1, wherein a metal radiating member is bonded to the optical communication integrated circuit, which is mounted on the electrical wiring substrate, using a radiating material.

9. The optical module according to claim 2, wherein the optical communication integrated circuit is bonded to the electrodes which are provided on the upper surface of the electrical wiring substrate.

10. The optical module according to claim 3, wherein the optical communication integrated circuit is bonded to the electrodes which are provided on the upper surface of the electrical wiring substrate.
